(19) United States
(12) Patent Application Publication
(10) Pub. No.: US 2020/0318142 A1
(43) Pub. Date: Oct. 8, 2020

(54) ALGAL BIOFUEL PRODUCTION AS AN AIR SEPARATION UNIT FOR SYNGAS, HYDROGEN, OR POWER PRODUCTION

(71) Applicant: ExxonMobil Research and Engineering Company, Annandale, NJ (US)

(72) Inventors: Everett J. O'Neal, Asbury, NJ (US); Patrick L. Hanks, Bridgewater, NJ (US); Anastasios I. Skoulidas, Pittstown, NJ (US); Anjaneya S. Kovvali, Herndon, VA (US); Sarah E. Feicht, Raritan, NJ (US)

(21) Appl. No.: 16/820,287

(22) Filed: Mar. 16, 2020

Related U.S. Application Data
(60) Provisional application No. 62/829,179, filed on Apr. 4, 2019.

(51) Int. Cl. C12P 3/00; C12M 1/00; C12F 3/02

(52) U.S. Cl. CPC C12P 3/00; C12F 3/02; C12M 29/00

(57) ABSTRACT

This invention relates to methods and apparatus for harvesting by-product oxygen from algae ponds or bioreactors (collectively, “algal biofuel production”) for use in an oxygen-requiring process that requires oxygen as a reactant such as syngas, hydrogen, or power production processes, which optionally can be integrated with the algal biofuel production. In some embodiments, the invention provides methods that include a method comprising: collecting oxygen from an algal biofuel production process; and using the collected oxygen in an oxygen-requiring process that requires oxygen as a reactant. In some embodiments, the invention provides systems that include an integrated system comprising: an algal bioreactor that produces biodiesel and oxygen, a pipeline for transporting oxygen to an oxygen-requiring process unit so that the oxygen can be used as reactant in the oxygen-requiring process unit, and the oxygen-requiring process unit.

### CROSS-REFERENCE TO RELATED APPLICATIONS

This application claims priority to U.S. Provisional Patent Application Ser. No. 62/829,179, filed Apr. 1, 2019, which is incorporated herein by reference in its entirety.

### BACKGROUND OF THE INVENTION

This invention relates to methods and apparatus for harvesting oxygen that is produced as a by-product from algae ponds or bioreactors (collectively, “algal biofuel production”) for use in oxygen-requiring processes that requires oxygen as a reactant such as syngas, hydrogen, or to power production processes, which optionally can be integrated with the algal biofuel production.

Algaculture is used for producing renewable raw materials for biofuels. The vegetable oil from algae can be used directly (straight vegetable oil that is esterized into biodiesel) or refined into various biofuels, including renewable diesel and jet fuel, in addition to other chemical ingredients for products such as cosmetics. The carbohydrates (sugars) from algae can be fermented to make additional biofuels, including ethanol and butanol, as well as other products such as plastics and biochemicals. Biomass from algae can be used for pyrolysis oil or combined heat and power generation. Algae-derived renewable diesels and jet fuels are drop-in fuels that directly replace petroleum fuels without modification of engines. They meet all the specifications for the petroleum fuel they replace. The high lipid content, high growth rate and ability to rapidly improve strains and produce co-products, without competing for arable land, make algae an exciting addition to the sustainable fuel portfolio.

Algaculture generally involves growing algae in closed systems or open systems. Closed systems include both photobioreactors for photosynthetic algae strains and traditional bioreactors (enclosed tanks such as those used in other microbial growth) for those, such as cyanobacteria, that do not require sunlight. Photobioreactors and bioreactors are referred to herein as “bioreactors.” Open pond systems are used as well, but can be sensitive to various environmental factors, such as contamination by other algae strains, or variations in nutrients, heat and light. Further, the areas in which algae can be most effectively grown are limited (e.g., Saudi Arabia, Africa, South America, etc.) because of the sun and weather conditions present. Unfortunately, however, the availability of large-scale CO₂ for the algal process is limited in such areas.

During photosynthesis, green algae harvest solar energy and carbon dioxide to split water atoms, produce biomass feedstock, and release oxygen. This oxygen by-product is significant. In fact, a molecule of oxygen is produced for every molecule of carbon dioxide that is consumed in the process. For large-scale applications of algal biofuel production, this oxygen represents a significant potentially valuable by-product that has heretofore been ignored. For example, a 10 kbd algae facility can produce approximately 120 MMSCFD of oxygen, or nearly 5000 tpd, which corresponds to the amount of oxygen produced in a world-scale air separation plant. If the algal system is an open pond, the oxygen is just released. If it is a closed system, the oxygen is often vented off because too much oxygen can oxidize the algae (e.g., photobleach) and prevent growth. Thus, in closed systems especially there is an opportunity to capture and harvest (“collect”) this valuable oxygen for use in oxygen-requiring processes that require oxygen as a reaction product. Nevertheless, most innovation ignores this oxygen by-product and instead focuses on methods and apparatus to use carbon dioxide produced in industrial processes in algal biofuel production processes in order to reduce carbon emissions from those industrial processes. FIG. 1 describes an algal biofuel production process.

Oxygen is a necessary reaction product in many industries such as oxycombustion power, hydrogen generation, and syngas generation (collectively, “oxygen-requiring processes”). Such industries often require an expensive air separation unit to produce the oxygen necessary for the reactions. An air separation unit usually is upstream of the oxygen-requiring process, and separates atmospheric air into its primary components, typically nitrogen and oxygen, and sometimes also argon and other rare inert gases.

The most common commercial method for producing oxygen is the separation of air using either a cryogenic distillation process or a vacuum swing adsorption process. Oxygen generators are also available. However, such air separation systems and oxygen generators are very expensive and energy intensive in order to produce the oxygen necessary for syngas, hydrogen, or power production processes. For example, 500 tons of oxygen a day would feed a megawatt oxycombustion power plant.

Therefore, there is a need to take advantage of the by-products of algal production by providing apparatus and systems by which the oxygen by-product can be collected/harvested for use in subsequent oxygen-requiring processes such as those that typically require capital-intensive air separation units.

### SUMMARY OF THE INVENTION

This invention relates to methods and apparatus for harvesting by-product oxygen from algae ponds or bioreactors (collectively, “algal biofuel production”) for use in an oxygen-requiring process that requires oxygen as a reactant such as syngas, hydrogen, or power production processes, which optionally can be integrated with the algal biofuel production.

In some embodiments, the invention provides methods that include a method comprising: collecting oxygen from an algal biofuel production process; and using the collected oxygen in an oxygen-requiring process that requires oxygen as a reactant.

In some embodiments, the invention provides methods that include a method comprising: collecting oxygen from an algal biofuel production process; and using the collected oxygen in a power production process that requires oxygen as a reactant.

In some embodiments, the invention provides methods that include a method comprising: collecting oxygen from an algal biofuel production process; and using the collected oxygen in a syngas production process that requires oxygen as a reactant.

In some embodiments, the invention provides systems that include a system comprising: an algal biofuel bioreactor, the algae biofuel bioreactor producing biodiesel and oxygen; and a transportation means for transporting the oxygen to an oxygen-requiring process located downstream of the algal biofuel reactor.

In some embodiments, the invention provides systems that include an integrated system comprising: an algal bioreactor that produces biodiesel and oxygen, a pipeline for transporting oxygen to an oxygen-requiring process unit so that the oxygen can be used as a reactant in the oxygen-requiring process unit, and the oxygen-requiring process unit. In some embodiments, such integrated systems include a pipeline connecting the oxygen-requiring process unit to the algal bioreactor to transport by-products (e.g., CO₂, NON, etc.) from the oxygen-requiring process unit to the algal bioreactor.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is an illustration of an algae biofuel process.

FIG. 2 is an illustration of an integrated embodiment of this invention wherein an algal production process is integrated with an oxycombustion power process.

FIG. 3 is an illustration of an integrated embodiment of this invention wherein an algal production process is integrated with a syngas generation process.

FIG. 4 is an illustration of an integrated embodiment of this invention wherein an algal production process is integrated with an RFR process to produce power.

FIG. 5 illustrates a reactor system that includes two solids beds to transfer heat to and from gases flowing through the system, and includes one bed containing reforming catalyst between the solids beds.

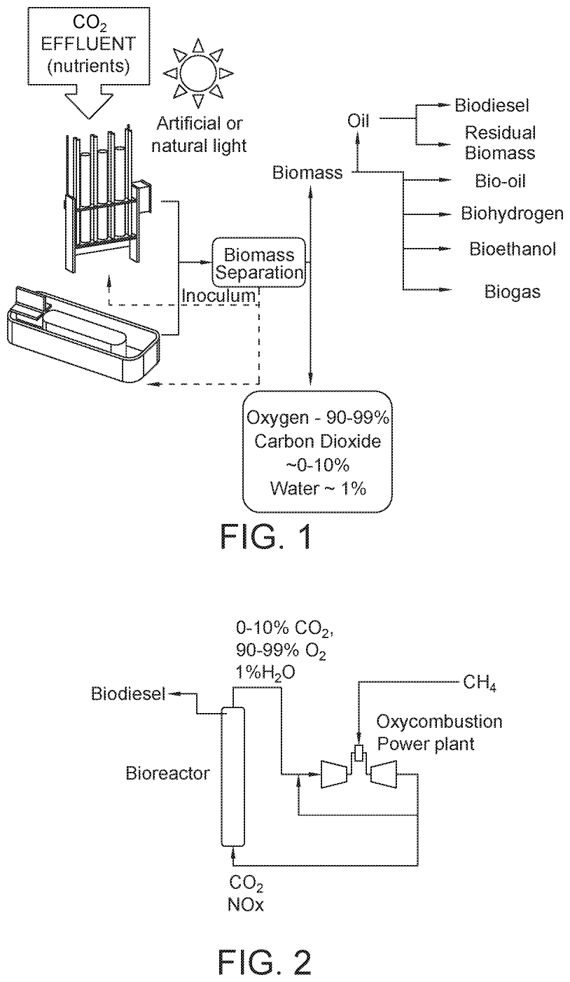

### DETAILED DESCRIPTION

This invention relates to methods and apparatus for harvesting by-product oxygen from algal biofuel production processes for use in oxygen-requiring processes that require oxygen as a reactant such as syngas, hydrogen, or power production processes. In certain preferred embodiments, this invention provides for the integration of algal biofuel production processes with oxygen-requiring processes such as syngas, hydrogen, or power production processes.

The inventions described in this disclosure provide several advantages. Of particular benefit is that the oxygen produced in algal biofuel production processes can be put to productive use in oxygen-requiring processes that require oxygen as a reactant instead of being vented to the atmosphere. The algal biofuel production process can thereby be a beneficial source of cheap oxygen for oxygen-requiring processes such as syngas, hydrogen, or power production processes. Further, expensive air separation units can be avoided for those oxygen-requiring processes.

Moreover, as an additional advantage, in some instances, the algal biofuel production process can take advantage of by-products of the oxygen-requiring process (e.g., CO₂, nitrogen compounds) for use in producing biofuels, which can help reduce emissions from the oxygen-requiring processes as well as our dependence on fossil fuels. The global production potential for microalgae biofuels depends on the resources available for algal cultivation, principally water, land and CO₂, and the local climatic conditions, which set potential productivity in gallons of oil produced per acre per year for each specific location. The ability to get nutrients from the oxygen-requiring processes for the algal biofuel production process de-constrains algae reactor locations around the world and broadens the possibly location where algae can be grown to produce biofuel. Proximity to a nearby flue gas CO₂ source is perhaps the single most restrictive criterion, because transport of flue-gas from power plants (˜10% CO₂ content) restricts suitable areas for algal biofuel production processes to about a 10 km radius around CO₂ point-sources. Further, the amount of CO₂ from each source is required to be at least 600 kilo tons per annum (ktpa), to support large-scale algal biofuels production. The integrated embodiments disclosed herein in particular describe the synergistic use of CO₂ to enable industrial scale biofuel production (e.g., 10,000 barrels per day of algal oil).

In some embodiments, the algal biofuel production process may be located at a distance from the oxygen-requiring process. The oxygen can be transported to the oxygen-requiring process by any suitable means including truck, train, pipeline, etc. Alternatively and advantageously, to save on transportation costs, the algal biofuel production can be located near enough to the syngas, hydrogen, or power production facility to deliver the oxygen by pipeline directly from the algal production facility to the oxygen-requiring process facility. Such embodiments are described herein as “integrated embodiments.”

In one embodiment, the oxygen-requiring process is a power production process such as an oxycombustion process. Oxycombustion involves burning a fuel (e.g., methane) using oxygen instead of air as the primary oxidant to produce power, along with other by-products such as carbon dioxide, water and nitrogen oxides. Oxygen collected from an algal biofuel production may be transported to the oxycombustion facility in any suitable manner including by pipeline, cylinder truck, tanker truck, train, etc. Optionally, the collected oxygen can be purified prior to transport to or placement in the oxygen-requiring process. Similarly, the by-products from the oxycombustion power plant (e.g., CO₂, NON) can be transported to the algal biofuel production facility for use therein.

Alternatively, the algae production process can be integrated with the oxycombustion power plant to obtain multiple synergistic benefits. In such an integrated system, oxygen can be directly fed to the oxycombustion power process from the algal biomass process, for example, via a pipeline. FIG. 2 illustrates an example of such an integrated process in which the algal biomass process includes a bioreactor. A bioreactor is integrated with an oxycombustion power plant so as to provide oxygen from the bioreactor to the oxycombustion plant.

As shown in FIG. 2, several synergies are obtained through this integration. First, the oxygen produced in the bioreactor is used in the oxy-combustion process to make power by reaction with a fuel (e.g., methane). In effect, the bioreactor becomes an air separation unit for the oxycombustion power plant, but with a valuable product produced, biodiesel. Oxygen combines with methane as the fuel in the oxycombustion power plant to produce power as well as certain by-products. The by-products produced in the oxycombustion plant, specifically the carbon dioxide, water, and nitrogen oxides (NOₓ), can be fed back to the bioreactor to be used in the algae bioreactor for production of biodiesel. For example, the NON can be cleaned up by the algae in the bioreactor as algae consume nitrogen as nitrate, which satisfies some of the nutrition requirements of the algae while allowing for higher flame temperatures in the oxycombustion plant. Although higher flame temperatures generally imply higher cycle efficiencies in power generation, such temperatures often generate more NON, which can be problematic for the combustion system.

In another embodiment, the oxygen-requiring process is a syngas (also known as synthesis gas) production process. Syngas can be produced from many sources, including natural gas, coal, biomass, or virtually any hydrocarbon feedstock, by reaction with steam (steam reforming), carbon dioxide (dry reforming), or oxygen (partial oxidation). According to this invention, if using a partial oxidation syngas process, an algal production process can be used as the source of oxygen for the syngas process. Oxygen collected from an algal biofuel production may be transported to the syngas production facility in any suitable manner including by pipeline, cylinder truck, tanker truck, train, etc. Optionally, the collected oxygen can be purified prior to transport to or placement in the syngas production facility.

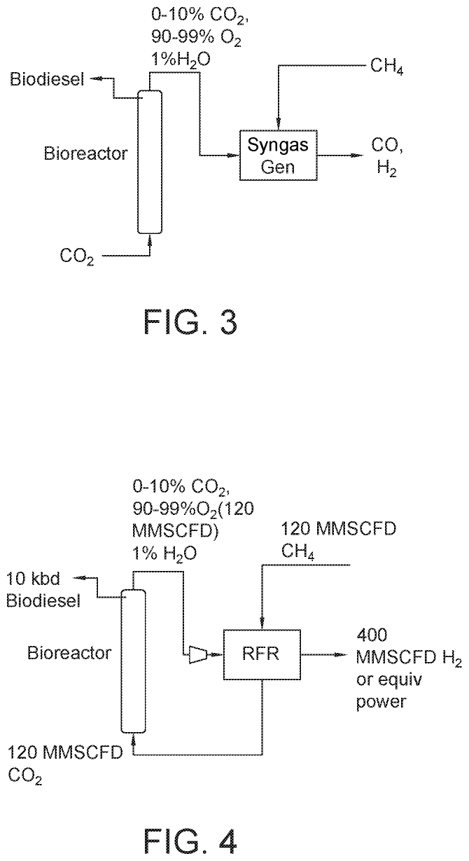

Optionally, the algal production facility can be integrated with a syngas generation facility. FIG. 3 illustrates a bioreactor as an algal production process from which oxygen, carbon dioxide, and water is produced in addition to biodiesel. The oxygen inter alia can be fed into the syngas generation facility with methane to produce syngas. The by-products from the syngas process, including carbon monoxide and hydrogen, can be used to synthesize fuels downstream of the syngas unit. In such an integrated embodiment, carbon dioxide for the algae in the bioreactor will need to be obtained from a source other than the syngas generation facility.

Optionally, a reforming technology (RFR) can be integrated with the algae bioreactor for hydrogen and power generation. FIG. 4 illustrates such an example with an approximate product rate for a 10 kbd algae facility. Oxygen produced in the bioreactor can be introduced into the RFR with a fuel such as methane to produce power. By-products from the RFR process such as CO₂ can be fed back to the bioreactor as a reactant therein. A significant synergy with this integrated embodiment is a performance increase in RFR regeneration associated with the leftover CO₂ residing in the oxygen stream, due to the improvement in heat capacity of the diluent stream. Higher heat capacities lower total regeneration mole flow rate, which reduces pressure drop in the reactor. Pressure drop though an RFR system is known to be a bottleneck in scale-up, and drives reactor size and cost.

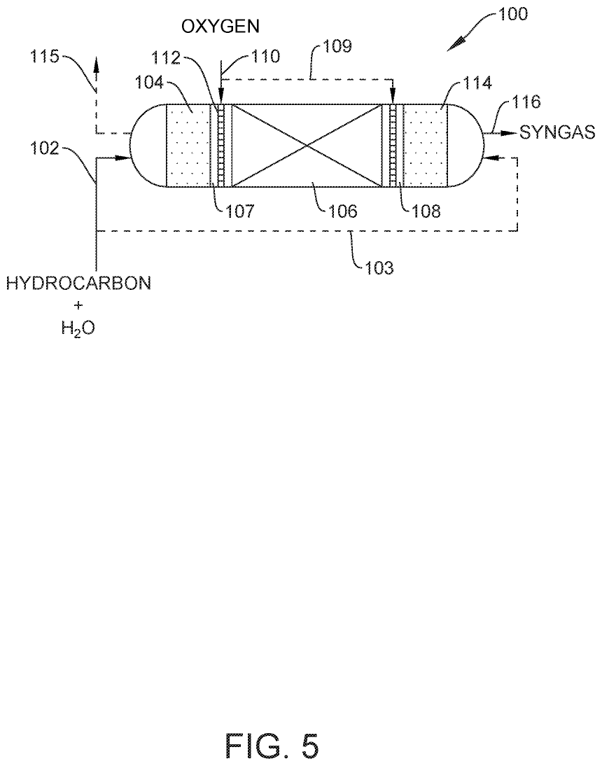

FIG. 5 illustrates another example of a process wherein oxygen from a bioreactor may be supplied as a reactant to a subsequent oxygen-requiring process. FIG. 5 from U.S. Pat. No. 7,740,829 illustrates oxygen being introduced into a reactor system that includes two solids beds to transfer heat to and from gases flowing through the system, and includes one bed containing reforming catalyst between the solids beds. Hydrocarbons and water are injected as a vapor into the reaction system at 100 through a line 102. The vapor passes through a zone in the reaction system that contains a bed of solids 104, with the bed being sufficiently hot to heat the vapor to at least about 900° C. The hot vapor is then flowed to an oxidation zone 107 where oxygen is introduced to oxidize a least a portion of the hydrocarbon in the reformed gas and form a synthesis gas. Oxygen from the bioreactor can be introduced to the oxidation zone 107 through a line 110 and distributed in the oxidation zone 107 by way of a distributor 112. Hot gas from the oxidation zone 107 is then sent to a zone 106 containing reforming catalyst. As the vapor flows through the bed of reforming catalyst, at least a portion of the hydrocarbon is converted to CO and CO₂.

Preferably, the regeneration step of the reformer (RFR) could be performed at a low pressure (blower only) to avoid expensive recompression of the biogas oxygen. Optionally, RFR can be run symmetrically as an ATR in both directions. The CO₂ can be recycled to the bioreactor. Integrating this embodiment of a RFR with an algae CO₂ source allows for syngas production for a GTL application, without any CO₂ recycle to the algae pond.

Embodiments disclosed herein include:

### Embodiment 1

A method comprising: collecting oxygen from an algal biofuel to production process; and using the collected oxygen in an oxygen-requiring process that requires oxygen as a reactant.

### Embodiment 2

The method according to Embodiment 1 wherein the algal biofuel production facility is an open pond system or a closed bioreactor system.

### Embodiment 3

The method according to Embodiment 1 further comprising purifying the collected oxygen.

### Embodiment 4

The method of Embodiment 1 wherein the oxygen-requiring process is an oxycombustion power plant, a syngas process, or a reforming power process.

### Embodiment 5

A method comprising: collecting oxygen from an algal biofuel production process; and using the collected oxygen in a power production process that requires oxygen as a reactant.

### Embodiment 6

The method of Embodiment 5 further comprising producing power from the power production process.

### Embodiment 7

The method of Embodiment 5 wherein the power production process is an oxycombustion power process.

### Embodiment 8

A method comprising: collecting oxygen from an algal biofuel production process; and using the collected oxygen in a syngas production process that requires oxygen as a reactant.

### Embodiment 9

The method of Embodiment 8 further comprising producing syngas from the syngas production process.

### Embodiment 10

A system comprising: an algal biofuel bioreactor, the algae biofuel bioreactor producing biodiesel and oxygen; and a transportation means for transporting the oxygen to an oxygen-requiring process located downstream of the algal biofuel reactor.

### Embodiment 11

The system of Embodiment 10 wherein the oxygen-requiring process is a power production process or a syngas production process.

### Embodiment 12

The system of Embodiment 10 wherein the oxygen-requiring process is an oxycombustion power production process.

### Embodiment 13

The system of Embodiment 10 wherein the transportation means comprises one selected from the group consisting of a pipeline, cylinder truck, tanker truck, train.

### Embodiment 14

The system of Embodiment 10 wherein the algal biofuel bioreactor and the oxygen-requiring process are integrated.

### Embodiment 15

An integrated system comprising: an algal bioreactor that produces biodiesel and oxygen, a pipeline for transporting oxygen to an oxygen-requiring process unit so that the oxygen can be used as reactant in the oxygen-requiring process unit, and the oxygen-ic) requiring process unit.

### Embodiment 16

The system of Embodiment 15 wherein the oxygen-requiring process production facility is a power production process, an oxycombustion power production process, a reforming process, or a syngas production process.

### Embodiment 17

The system of Embodiment 15 further comprising a pipeline connecting the oxygen-requiring process unit to the algal bioreactor to transport by-products from the oxygen-requiring process unit to the algal bioreactor.

### Embodiment 18

The system of Embodiment 16 wherein the by-products from the oxygen-requiring process unit include carbon dioxide and/or nitrogen compounds.

By way of non-limiting example, exemplary combinations include: Embodiment 2 with Embodiment 3; Embodiment 2 with Embodiment 4; Embodiment 2 with Embodiment 5; Embodiment 6 with Embodiment 7; Embodiment 6 with Embodiment 8; Embodiment 8 with Embodiment 9; Embodiment 13 with Embodiment 14; Embodiment 15 with Embodiment 16; and Embodiment 15 with Embodiment 17 or 18.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A method comprising:
collecting oxygen from an algal biofuel production process; and
using the collected oxygen in an oxygen-requiring process that requires oxygen as a reactant.

2. The method according to claim 1 wherein the algal biofuel production facility is an open pond system or a closed bioreactor system.

3. The method according to claim 1 further comprising purifying the collected oxygen.

4. The method of claim 1 wherein the oxygen-requiring process is an oxycombustion power plant, a syngas process, or a reforming power process.

5. A method comprising:
collecting oxygen from an algal biofuel production process; and
using the collected oxygen in a power production process that requires oxygen as a reactant.

6. The method of claim 5 further comprising producing power from the power production process.

7. The method of claim 5 wherein the power production process is an oxy combustion power process.

8. A method comprising:
collecting oxygen from an algal biofuel production process; and
using the collected oxygen in a syngas production process that requires oxygen as a reactant.

9. The method of claim 8 further comprising producing syngas from the syngas production process.

10. A system comprising:
an algal biofuel bioreactor, the algae biofuel bioreactor producing biodiesel and oxygen; and
a transportation means for transporting the oxygen to an oxygen-requiring process located downstream of the algal biofuel reactor.

11. The system of claim 10 wherein the oxygen-requiring process is a power production process or a syngas production process.

12. The system of claim 10 wherein the oxygen-requiring process is an oxycombustion power production process.

13. The system of claim 10 wherein the transportation means comprises one selected from the group consisting of a pipeline, cylinder truck, tanker truck, train.

14. The system of claim 10 wherein the algal biofuel bioreactor and the oxygen-requiring process are integrated.

15. An integrated system comprising:
an algal bioreactor that produces biodiesel and oxygen,
a pipeline for transporting oxygen to an oxygen-requiring process unit so that the oxygen can be used as a reactant in the oxygen-requiring process unit, and
the oxygen-requiring process unit.

16. The system of claim 15 wherein the oxygen-requiring process production facility is a power production process, an oxycombustion power production process, a reforming process, or a syngas production process.

17. The system of claim 15 further comprising a pipeline connecting the oxygen-requiring process unit to the algal bioreactor to transport by-products from the oxygen-requiring process unit to the algal bioreactor.

18. The system of claim 16 wherein the by-products from the oxygen-requiring process unit include carbon dioxide and/or nitrogen compounds.
